SEQUENCE LISTING (2 sequences)

SEQ ID NO: 1 - protein, 8 aa, Artificial Sequence
  Model Peptide
SIINFEKL

SEQ ID NO: 2 - protein, 9 aa, Artificial Sequence
  Coronavirus spike epitope
QYIKWPWYI

(19) United States
(12) Patent Application Publication
(10) Pub. No.: US 2023/0033582 A1
(43) Pub. Date: Feb. 2, 2023

(54) POLYMERIC NANOPARTICLES FOR ENHANCED CANCER TREATMENT

(71) Applicant: THE QUEEN'S UNIVERSITY OF BELFAST, Belfast (GB)

(72) Inventors: Christopher SCOTT, Lisburn (GB); Daniel LONGLEY, Lisburn (GB); Luke HUMPHREYS, Oxfordshire (GB); William MCDAID, Manchester (GB)

(21) Appl. No.: 17/788,671

(22) PCT Filed: Dec. 24, 2020
(86) PCT No.: PCT/EP2020/087888

§ 371 Date: Jun. 23, 2022

(30) Foreign Application Priority Data
Dec. 24, 2019 (GB) 1919316.8

(51) Int. Cl. A61K 39/145; A61K 39/215; A61K 39/245; A61K 9/51; A61K 38/05; A61K 31/4709; A61K 31/47; A61K 33/06; A61K 31/4439; A61K 31/513; A61K 31/282; A61K 31/4745; A61K 31/519; A61K 31/7068; A61K 39/395; A61P 35/00

(52) U.S. Cl. CPC A61K 39/145; A61K 39/215; A61K 39/245; A61K 9/5153; A61K 38/05; A61K 31/4709; A61K 31/47; A61K 33/06; A61K 31/4439; A61K 31/513; A61K 31/282; A61K 31/4745; A61K 31/519; A61K 31/7068; A61K 39/3955; A61P 35/00; A61K 2039/585

(57) ABSTRACT

There is provided polymeric nanoparticles with non-tumour antigen payloads for use in tagging cells for destruction by a subject's immune system and the use thereof for the treatment of cancer. Suitably there is provided a method of treatment comprising administration of nanoparticle comprising a non-tumour protein payload to a subject with cancer, in particular to a cancer cell.

### FIELD OF THE INVENTION

There is provided polymeric nanoparticles with non-tumour antigen payloads for use in tagging cells for destruction by a subject's immune system and the use thereof for the treatment of cancer.

### BACKGROUND

Functional nanoparticles that can deliver drugs to sites within the body are known. Further nanoparticles have been used to provide tumour associated antigen to the body to provide for targeted stimulation of T cells involved in cancer.

WO2019/023622 discusses polymeric nanoparticles including payloads to provide for targeted stimulation of non-responsive T cells involved in cancer.

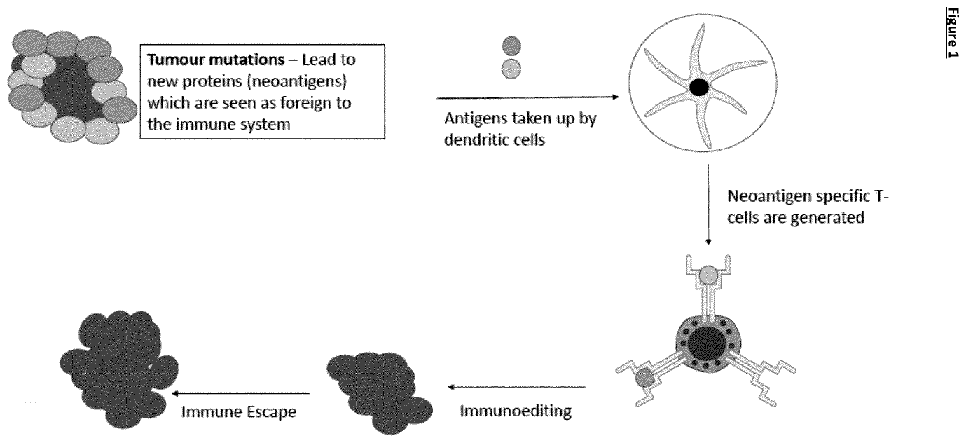

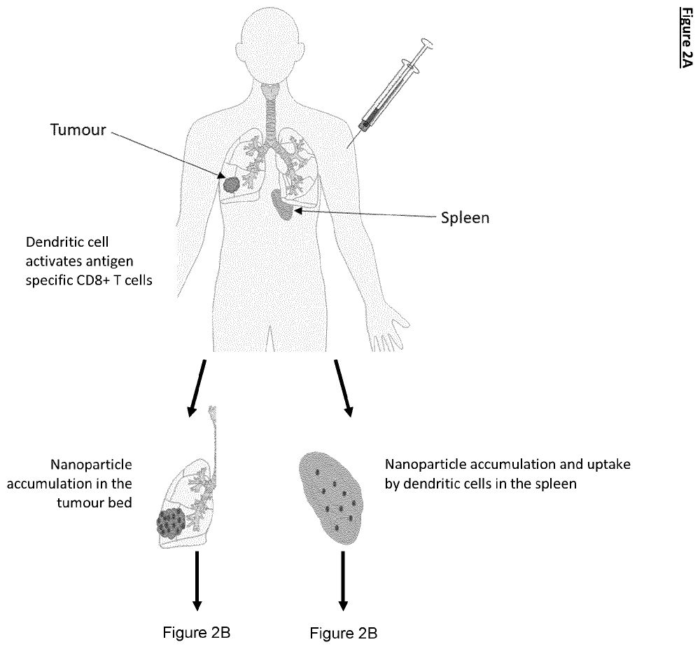

In existing tumour vaccine production, the neoantigens of a tumour are utilised to provide neoantigen specific T cells which can target the tumour—see FIG. 1. Additionally, dendritic cell-based cancer vaccines are generally known, which require the patient to undergo leukophoresis. Moreover, Chimeric Antigen Receptor (CAR)-T therapy is generally known, where T cells are engineered specific to the subject to recognise a specific protein from the cancer in question.

However, to generate vaccines using such neoantigens to stimulate the T cells requires the specific tumour to be analysed (requiring biopsy of the tumour), the HLA haplotype to be considered and whether the neoantigen will be presented such that it is sufficiently immunogenic. Moreover, the avidity of T-cell receptors generally lower for neoantigens than it is for foreign antigens.

### SUMMARY OF THE INVENTION

It would be desirable to provide a more effective way to target with high specificity, strong reactions to tumours by making them present a foreign (non-tumour neoantigen) antigen rather than a neoantigen.

The inventors have determined a method of ‘tagging’ tumour cells to allow them to be selectively targeted by the immune system. Suitably this ‘tagging’ is by providing a cancer cell with an immunogenic protein or peptide with is not neoantigen. In particular, the inventors have determined the use of a non-tumour protein payload delivered to a cancer cell in a nanoparticle, suitably via leaky blood vessels connected to the tumour, allows HLA presentation of at least a portion of the protein payload by the tumour cell and an immune response to be provided against the HLA presented protein. Suitably the nanoparticle may be a polymeric nanoparticle. In embodiments, the non-tumour payload may be provided by a non-naturally derived synthetic peptide. In embodiments the payload may be provided by a viral protein.

According to a first aspect of the invention, there is provided a method of treatment comprising administration of nanoparticle comprising a non-tumour protein payload to a subject with cancer.

Suitably the non-tumour protein, or peptide payload may be provided by a synthetic non-tumour, non-viral protein.

Optionally, the nanoparticle may have a size, i.e. a diameter, of between approximately 100 nm and approximately 300 nm, preferably between approximately 150 nm and approximately 250 nm, most preferably approximately 200 nm.

This provides the advantage that localisation of the nanoparticle also occurs in the spleen to engage dendritic cells.

Optionally, the nanoparticle may have a negative charge. In alternative aspects, the nanoparticle may have a positive charge, thereby enhancing antigen presentation. Suitably the non-tumour protein payload is encapsulated by the nanoparticle.

Advantageously, the present invention allows expression of a selected non-tumour payload protein antigen and does not thus require biopsy, or determination of specific tumour neoantigen(s), and is independent of the subject to be treated. Further, the composition and methodology of the present invention allow bispecific targeting of tumours and dendritic cells, without haplotype restriction.

Suitably the nanoparticle may be a polymeric, optionally the nanoparticle may be comprised of a biodegradable polymer. In some embodiments the biodegradable polymer is one or more of lactic acid polymers (PLA), glycolic acid polymers (PLG) and poly(lactic-co-glycolic) acid and (PLGA). Suitably a combination of polymers may be provided.

In some embodiments the non-tumour payload comprises at least one protein from an infectious disease. This can be advantageous as a subject may have an existing immune response to the infectious disease. In some embodiments the non-tumour, suitably encapsulated, payload can comprise two or more proteins. Suitably the payload may be any immunogenic protein or peptide to which a subject may have natural immunity. Suitably the immunogenic protein or peptide may be from a coronavirus, for example a corona virus spike protein. Suitably the payload may be a dominant or subdominant SARS-CoV-2 HLA class I and HLA-DR peptide in COVID-19 (for example as discussed Nelde, A., Bilich, T., Heitmann, J. S. et al. SARS-CoV-2-derived peptides define heterologous and COVID-19-induced T cell recognition. Nat Immunol 22, 74-85 (2021) published 30 Sep. 2020). Suitably the payload may be the peptide QYIKWPWYI (SEQ ID NO: 2). Suitably the proteins may be selected from an influenza virus, including Influenza A, B and/or C, a haemagglutinin and a neuraminidase. Suitably, one, two, three or more proteins may be provided. Suitably the non-tumour protein, or peptide payload may be optionally selected from;
(a) Influenza virus coat protein, for example derived from a serotypes including: A B and C but excluding D
(b) a haemagglutinin subtype or neuraminidase subtypes and/or

(c) a coat protein of the herpesviridae family, optionally Epstein Barr Virus proteins comprising constituents of the gHgLgp42 complex and LMP2A.

Suitably combinations of non-tumour protein or peptide payloads may be provided. In some embodiments, a polymeric nanoparticle that comprises an encapsulated payload of protein can increase in the expression of HLA on dendritic cells. In some embodiments, the activation and maturation of tumour dendritic cells and/or macrophages caused by the nanoparticles can lead them to efficiently present antigen to T lymphocytes, thus initiating an adaptive immune response. Suitably the uptake of the nanoparticle by the tumour cell(s) is by passive means, for example where cancer cells update more nanoparticles than typical cells in the subject (whilst the liver may uptake such nanoparticles, in such cases it is considered the liver will be able to recover from any response). Suitably the nanoparticles may be targeted to a cancer cell, for example using an antibody approach. Suitably the polymeric nanoparticle may be provided with a tumour cell binding member. Suitably the tumour cell binding member causes the polymeric nanoparticle to be preferentially targeted to a tumour cell.

In some embodiments, nanoparticles of the invention, such as polymeric nanoparticles of the invention, are administered in a combination therapy with an effective dose of an immunoregulatory agent, e.g. an antibody that binds to and inhibits activity of an immune checkpoint protein, a purified tumour antigen, e.g. a soluble tumour associated protein or fragment thereof, a cytokine, an antibody that binds to and inhibits activity of a T cell checkpoint protein, for example, but not limited to PD1; PD-L1; cytotoxic T-lymphocyte-associated protein 4 (CTLA-4); B- and T-lymphocyte attenuator (BTLA); lymphocyte activation gene 3 (LAG3); T-cell immunoglobulin and mucin domain 3 (TIM-3); V-domain Immunoglobulin Suppressor of T cell Activation; and T cell immunoreceptor with Ig and ITIM domains (TIGIT).

Suitably the non-tumour associated payload may be provided in combination with a lysosome perturbation agent, optionally wherein the lysosome perturbation agent is selected from at least one of Legumain or Cathepsin inhibitors, Alum, Omeprozole, Mefloquine, Tafenoquine, and Leu-Leu-OMe.

Suitably the non-tumour protein/peptide payload may be provided to allow MHC/HLA presentation of the peptide or of at least part of the protein payload to T cells. Suitably the nanoparticle may act to promote the delivery and presentation of the non-tumour protein/peptide payload. Suitably the nanoparticle may comprise additional agents either within or in combination with the nanoparticle to enhance the presentation of the non-tumour protein/peptide payload. For example, such agents may decrease or inhibit cleavage of the non-tumour protein/peptide payload and thus enhance the presentation of the protein/peptide payload. Suitably such agents may be lysosomal disruption agents. Suitably L-leucyl-L-leucine methyl ester (LLOMe) may be provided to enhance non-tumour protein/peptide payload presentation. Suitably an additional agent may be selected from Mefloquine, gencitibine or the like.

In embodiments, methods are provided for treatment of a cancer, which cancer is a solid tumour. In embodiments a solid tumour can be one of a carcinoma, sarcoma, lymphoma, myeloma, cancers of the central nervous system, e.g. glioma, medulloblastoma, or astrocytoma.

In embodiments a method of treatment is provided comprising: providing an individual with an effective dose of polymeric nanoparticles described herein, wherein the effective dose provides for activation of tumour-specific CD8⁺ T cells, and reduces the growth of tumour cells by increased targeted killing of tumour cells.

In embodiments a pharmaceutical formulation is provided, e.g. for use in the treatment of a human subject, where the formulation comprises a nanoparticle as described herein, as a unit dose, e.g. as a sterile pre-pack in a unit dose with diluent for intravenous delivery device. Pharmaceutical compositions or kits may further comprise a second active agent, e.g. a chemotherapeutic.

Preferred features and embodiments of each aspect of the invention are as for each of the other aspects mutatis mutandis unless context demands otherwise.

Each document, reference, patent application or patent cited in this text is expressly incorporated herein in their entirety by reference, which means it should be read and considered by the reader as part of this text. That the document, reference, patent application or patent cited in the text is not repeated in this text is merely for reasons of conciseness.

Reference to cited material or information contained in the text should not be understood as a concession that the material or information was part of the common general knowledge or was known in any country.

Throughout the specification, unless the context demands otherwise, the terms ‘comprise’ or ‘include’, or variations such as ‘comprises’ or ‘comprising’, ‘includes’ or ‘including’ will be understood to imply the includes of a stated integer or group of integers, but not the exclusion of any other integer or group of integers.

Embodiments of the invention will now be described with reference to the accompanying figures by way of example only in which:

FIG. 1 illustrates production of neoantigen specific T cells;

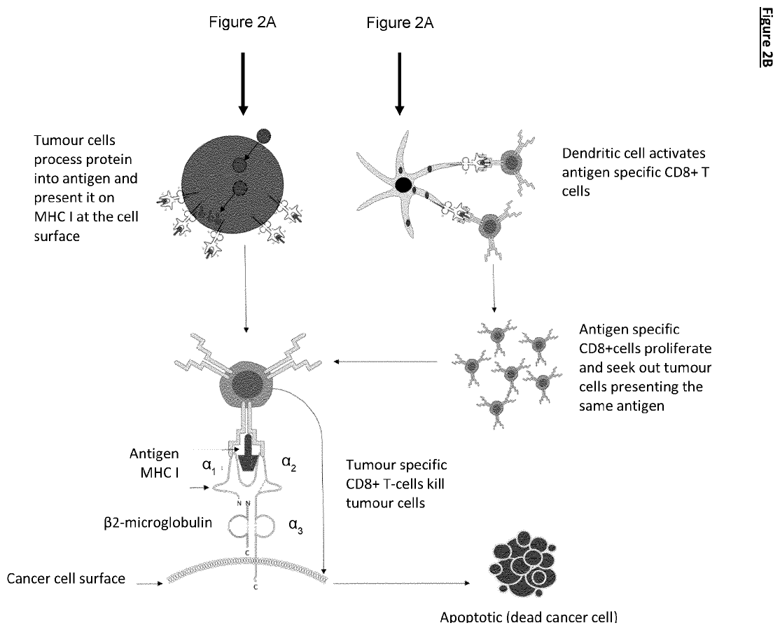

FIG. 2 illustrates a method of the present invention; an antigen encapsulated nano-formulation is administered to a patient. This formulation is targeted to both the dendritic cells in the spleen and tumour cells where the nanoparticles are taken up. Dendritic cells process and present the antigen to CD8+ T cells which become activated and seek out cells presenting the same antigenic proteins. Meanwhile, tumour cells also processed and present the same antigen on the surface making the tumour cells visible to antigen-activated CD8+ T cells. CD8+ T cells elicit a cytotoxic attack on tumour cells leading to tumour cell death.

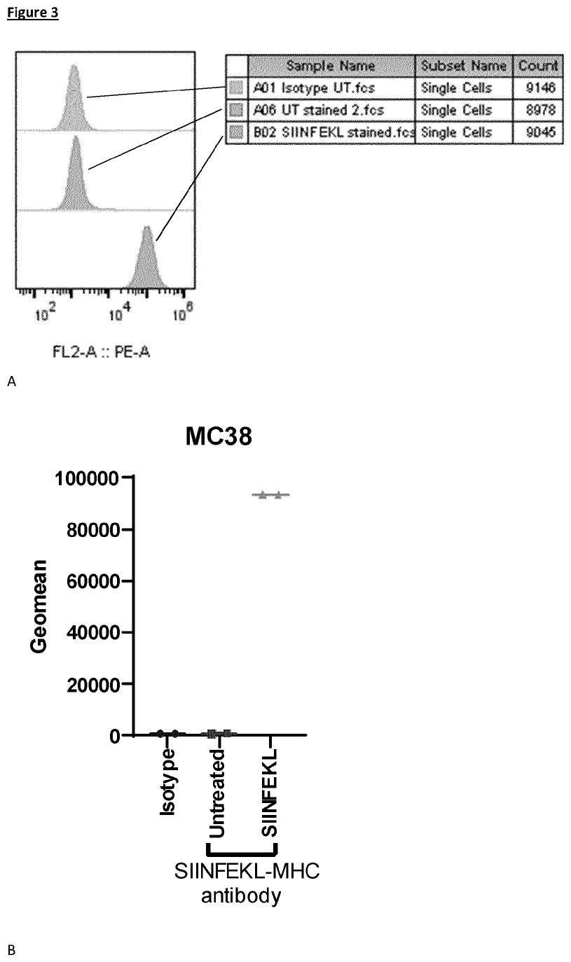

FIG. 3 illustrates Validation of MHC:SIINFEKL antibody: MC38 cells were treated with either PBS or SIINFEKL peptide and incubated for 4 hours. Cells were then incubated with SIINFEKL antibody or an IgG control and antigen presentation was determined by flow cytometry, in particular MC38 cells were treated with either PBS or 1 μM SIINFEKL peptide for 4 hours. Cells were stained with SIINFEKL-bound MHC antibody and SIINFEKL presentation was determined by flow cytometry. An Isotype specific IgG control (SIINFEKL IgG) was used to detect non-specific binding of our antibody. B The SIINFEKL-bound MHC antibody detected high levels SIINFEKL on the surface of SIINFEKL-treated cells leading to a strong fluorescent signal (geomean). This signal was not seen in cells which were not treated with SIINFEKL confirming that the antibody is functional. No signal was seen in cells stained with the isotype specific antibody suggested that the SIINFEKL-bound MHC antibody is specific and does not bind to random artefacts.

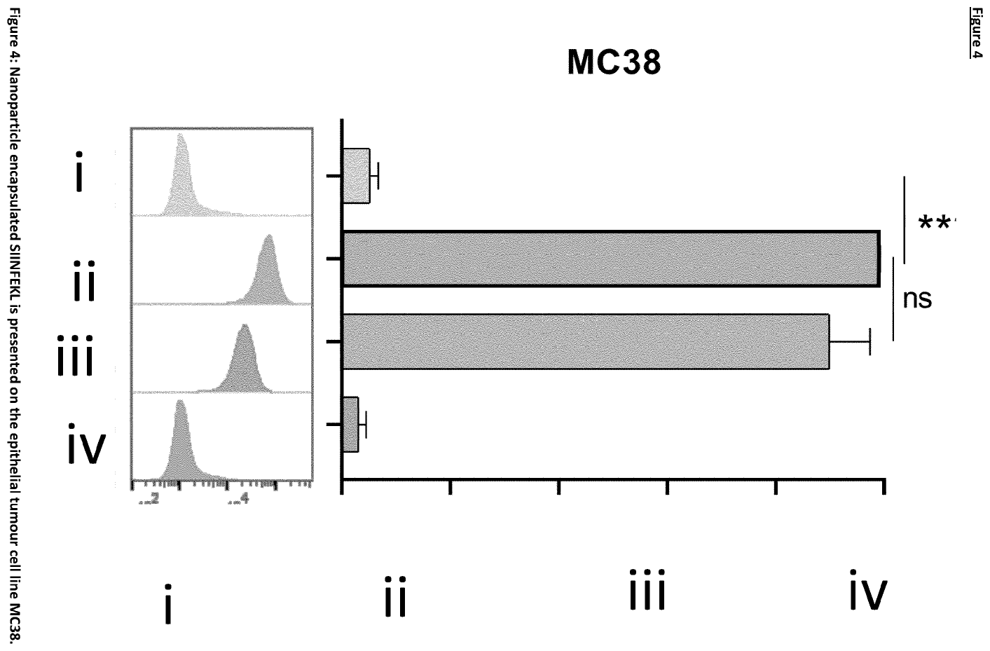

FIG. 4: illustrate the results of Nanoparticle encapsulated SIINFEKL is presented on the epithelial tumour cell line MC38: MC38 cells were treated with either 250 μg/mL blank nanoparticles (B-NP), 250 μg/mL nanoparticles encapsulating the SIINFEKL peptide (SIINFEKL-NP) or 1 μM free SIINFEKL (positive control) for 6 hours. Cells were stained with SIINFEKL-bound MHC antibody and SIINFEKL presentation was determined by flow cytometry. An Isotype specific IgG control (SIINFEKL IgG) was used to detect non-specific binding of our antibody;

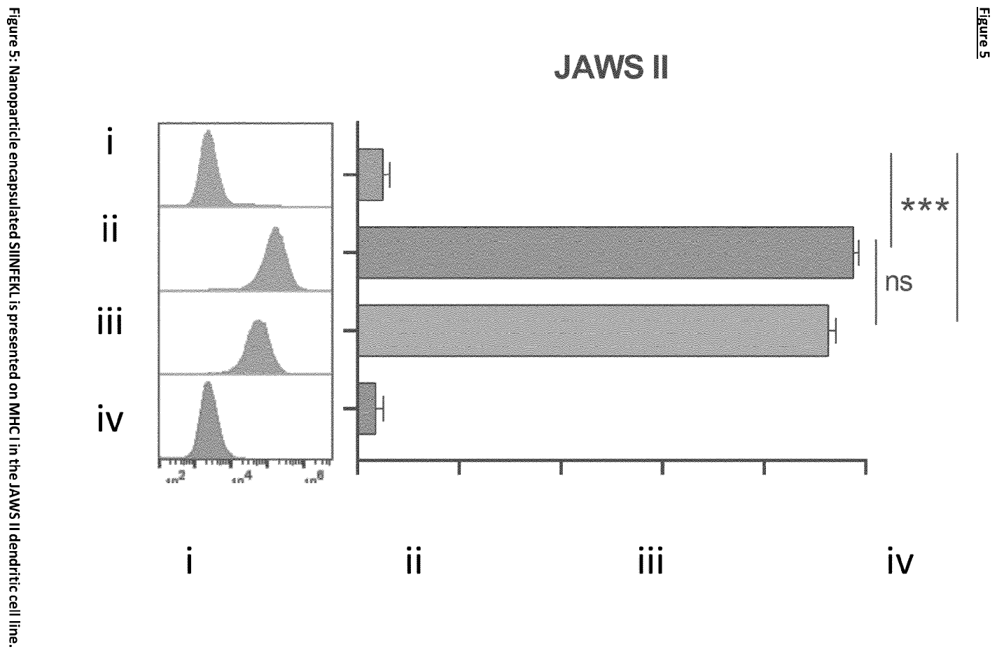

FIG. 5: illustrate the results of Nanoparticle encapsulated SIINFEKL is presented on MHC I in the JAWS II dendritic cell line: JAWS II cells were seeded in 6 well plates at 1×10⁵ and left overnight. The following day, cells were treated with either 500 μg of PLGA-SIINFEKL NP (SIINFEKL NP), PLGA NP (B-NP) or free SIINFEKL (Positive control). The cells were incubated at 37° C. in 5% CO₂ for 6 hours. After incubation, an antibody specific for SIINFEKL bound to MHC I was used to detect cell surface expression of the antigen. Assessment was undertaken using flow cytometry. An Isotype specific IgG control (SIINFEKL IgG) was used to detect non-specific binding of the antibody. Accordingly, B-NP and SIINFEKL IgG treated cells were negative for antibody staining, whereas the positive control showed clear positivity in almost 100% of cells. Importantly, the SIINFEKL-NP treated cells also showed positivity, strongly suggesting that these cells were expressing SIINFEKL bound to MHC class 1. Diagram is a representative figure from 3 independent experiments;

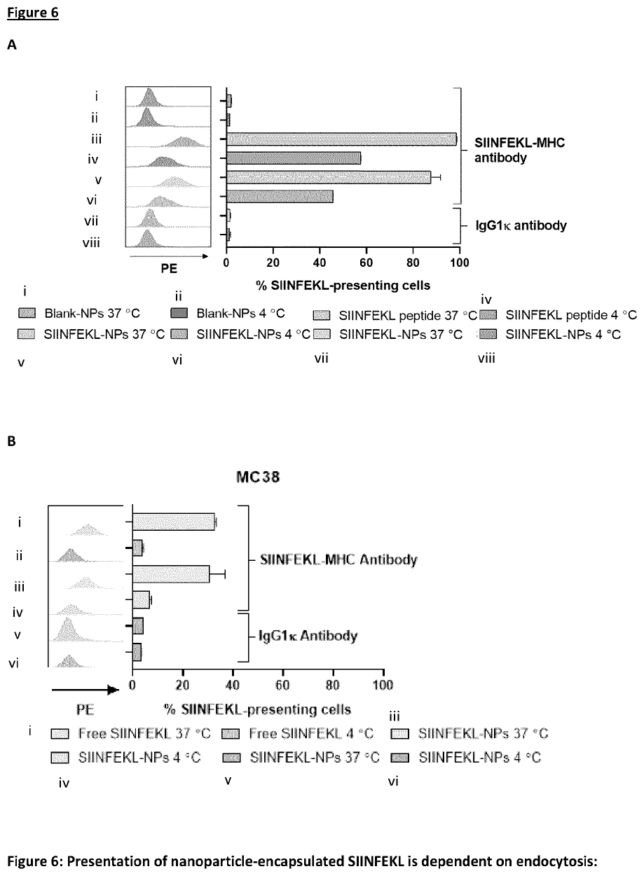

FIG. 6: illustrate the results of Presentation of nanoparticle-encapsulated SIINFEKL is dependent on endocytosis: (A) JAWS II cells and (B) MC38 cells ells were treated with either 250 μg/mL blank nanoparticles (B-NP), 250 μg/mL nanoparticles encapsulating the SIINFEKL peptide (SIINFEKL-NP) or 1 μM free SIINFEKL (positive control) for 1 hour at either 4° C. or 37° C. Cells were washed to remove non-endocytosed nanoparticles or free peptide and were incubated at 37° C. Cells were stained with SIINFEKL-bound MHC antibody and SIINFEKL presentation was determined by flow cytometry. An Isotype specific IgG control (SIINFEKL IgG) was used to detect non-specific binding of our antibody. Incubation at 4° C. reduces the rate of endocytosis therefore nanoparticle delivery and SIINFEKL presentation is decreased.

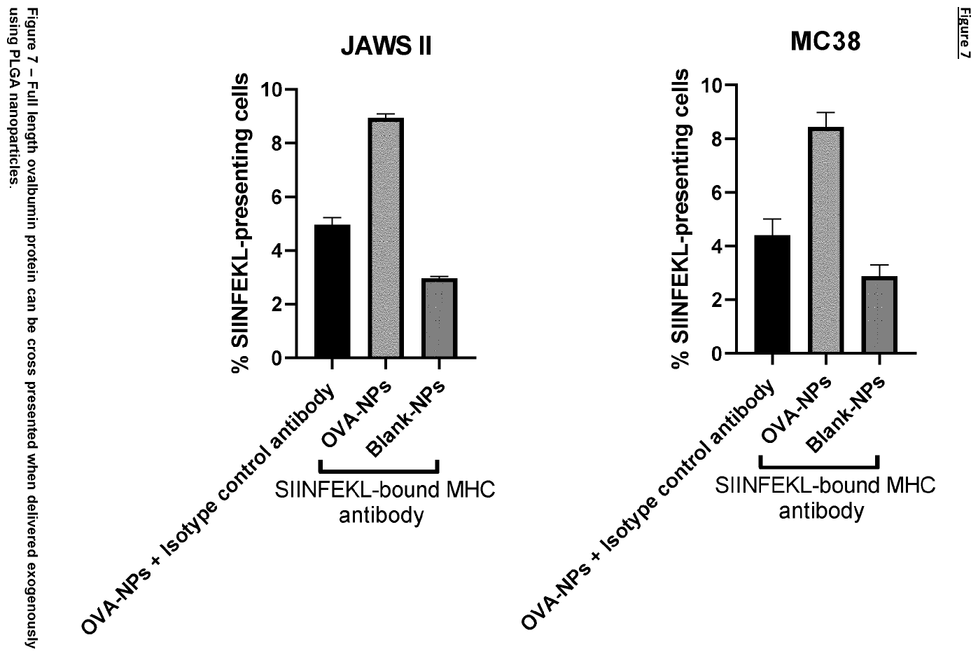

FIG. 7: illustrate the results of Full length ovalbumin protein can be cross presented when delivered exogenously using PLGA nanoparticles. Cells were treated with 250 μg/mL of either blank nanoparticles (B-NP), free SIINFEKL model antigen (Free SIINFEKL) or Ovalbumin encapsulated in PLGA NP for 24 hours. An IgG control was used to account for non-specific staining and a positive control was used to confirm positivity. Cells were incubated with a SIINFEKL-MHC I specific antibody and results assessed using flow cytometry. IgG was used as a negative control;

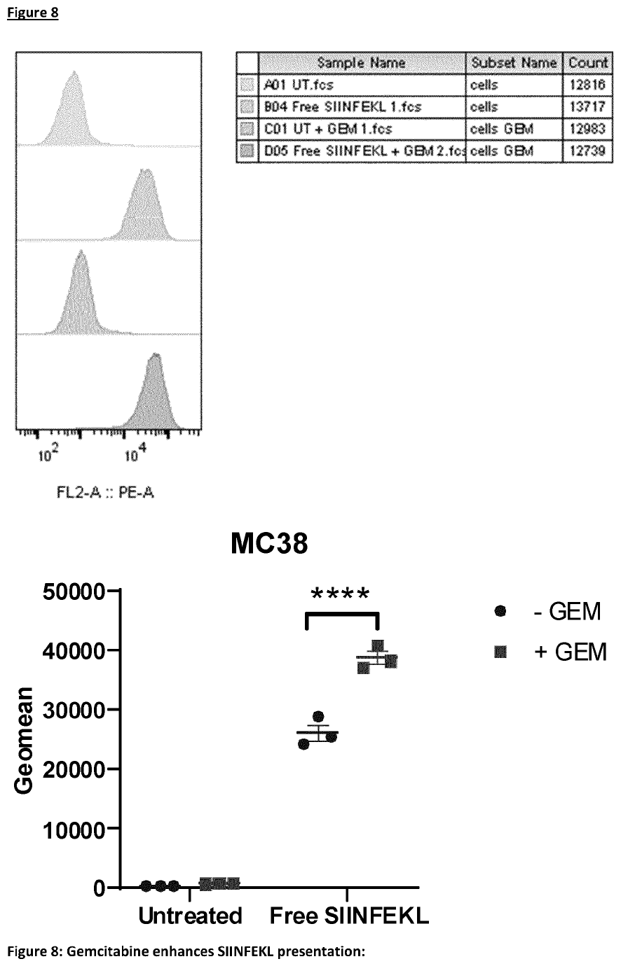

FIG. 8: illustrate the results of Gemcitabine enhances SIINFEKL presentation: MC38 cells were pretreated with 30 nm Gemcitabine for 24 hours. Then, cells were treated with 1 μM SIINFEKL peptide for 6 hours. After 6 hours, cells were stained with PE-SIINFEKL-bound MHC antibody to determine the levels of SIINFEKL presentation. Presentation of SIINFEKL was enhanced after pre-treatment with Gemcitabine highlighting the potential of boosting CD8+ T cell recognition of tumour cells through combining the nanoformulation with Gemcitabine;

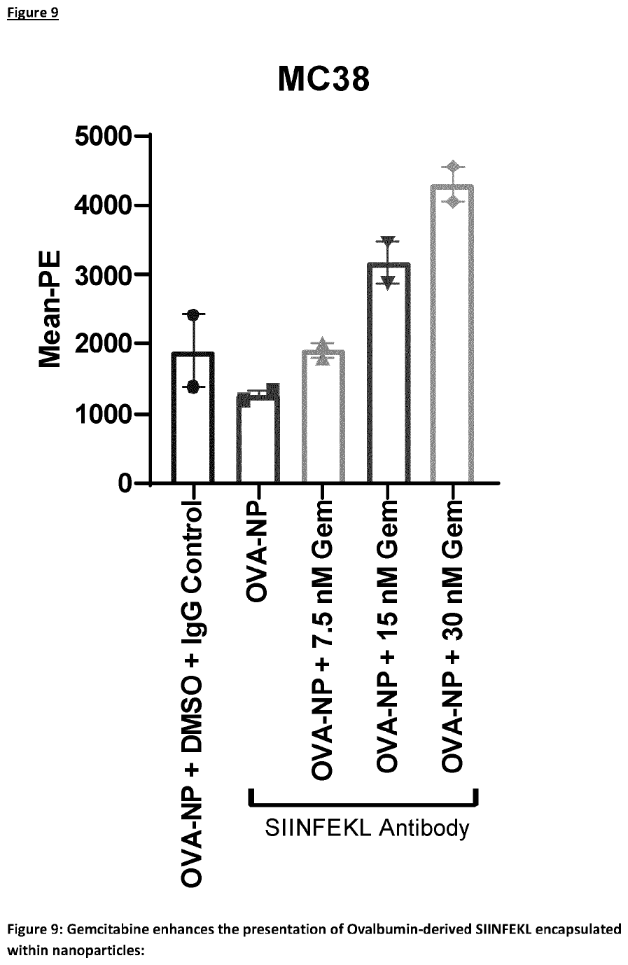

FIG. 9: illustrate the results of Gemcitabine enhances the presentation of Ovalbumin-derived SIINFEKL encapsulated within nanoparticles: MC38 cells were pre-treated with 7.5, 15 and 30 nm Gemcitabine for 24 hours. Then, the cells were treated with OVA-NPs for 6 hours. After 6 hours, cells were stained with PE-SIINFEKL-Bound MHC antibody and an isotype control antibody to determine levels of OVA-derived SIINFEKL presentation. NP-encapsulated OVA-derived SIINFEKL was seen to be enhanced after pre-treatment with Gemcitabine;

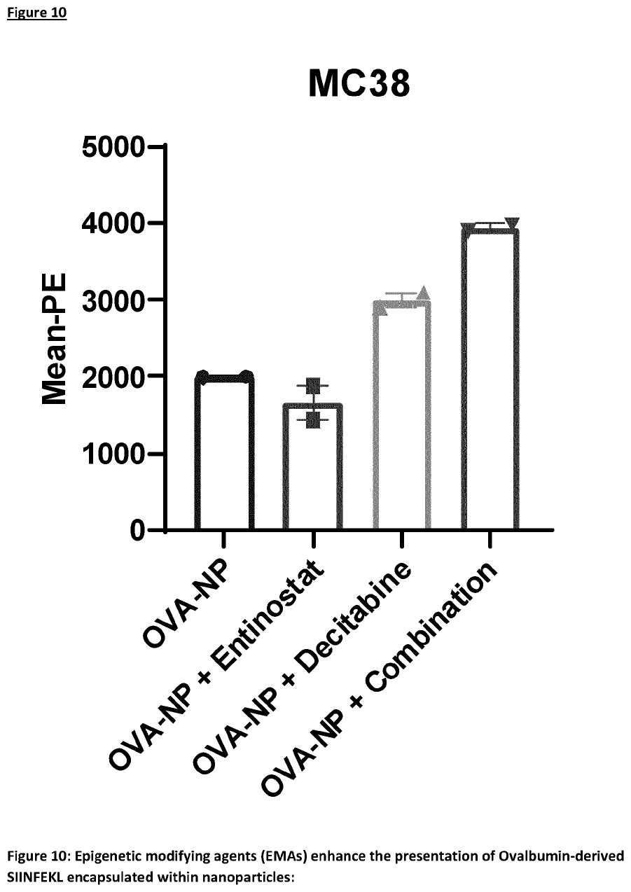

FIG. 10: illustrate the results of Epigenetic modifying agents (EMAs) enhance the presentation of Ovalbumin-derived SIINFEKL encapsulated within nanoparticles: MC38 cells were pre-treated with Entinostat and Decitabine. After 24 hours, cells were treated with OVA-NPs (250 μg/mL) for 6 hours. Cells were stained with SIINFEKL-bound MHC antibody and the surface presentation of SIINFEKL was determined by flow cytometry. Both agents in combination enhanced SIINFEKL presentation;

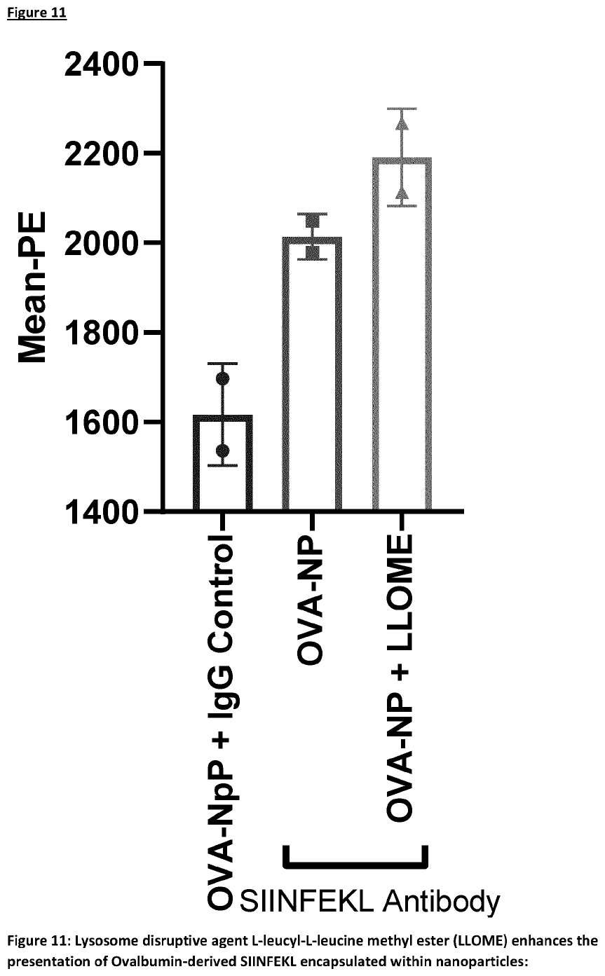

FIG. 11: illustrate the results of Lysosome disruptive agent L-leucyl-L-leucine methyl ester (LLOME) enhances the presentation of Ovalbumin-derived SIINFEKL encapsulated within nanoparticles: MC38 cells were pre-treated with LLOME for 24 hours. Then, the cells were treated with OVA-NPs for 6 hours. After 6 hours, cells were stained with PE-SIINFEKL-bound MHC antibody and an isotype control antibody to determine levels of OVA-derived SIINFEKL presentation. NP-encapsulated OVA-derived SIINFEKL was seen to be enhanced after pre-treatment with LLOME;

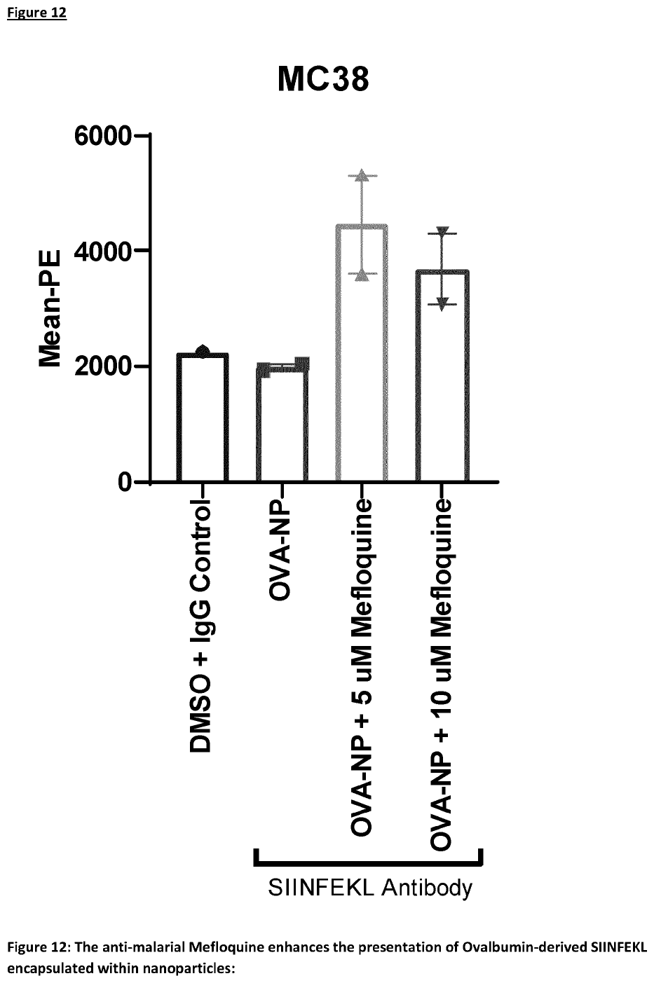

FIG. 12: illustrate the results of the anti-malarial Mefloquine enhances the presentation of Ovalbumin-derived SIINFEKL encapsulated within nanoparticles: MC38 cells were pre-treated with Mefloquine for 24 hours. Then, the cells were treated with OVA-NPs for 6 hours. After 6 hours, cells were stained with PE-SIINFEKL-bound MHC antibody and an isotype control antibody to determine levels of OVA-derived SIINFEKL presentation. NP-encapsulated OVA-derived SIINFEKL was seen to be enhanced after pre-treatment with Mefloquine.

### DETAILED DESCRIPTION OF THE INVENTION

As used herein the singular forms “a”, “and”, and “the” include plural meanings unless the context clearly dictates otherwise. All technical and scientific terms used herein have the same meaning as commonly understood to one of ordinary skill in the art to which this invention belongs unless clearly indicated otherwise.

Nanoparticles described herein comprise a payload for example an encapsulated payload, where the term payload refers to one, two, three or more non-tumour proteins contained within the nanoparticle. In some embodiments the encapsulated payload comprises at least one non-tumour protein. In some embodiments the encapsulated payload comprises two non-tumour proteins. In some embodiments the encapsulated payload comprises three non-tumour proteins.

The term “polypeptide,” “peptide,” “oligopeptide,” and “protein,” are used interchangeably herein, and refer to a polymeric form of amino acids of any length, which can include coded and non-coded amino acids, chemically, or biochemically modified or derivatized amino acids, and polypeptides having modified peptide backbones.

As used herein, the terms “purified” and “isolated” when used in the context of a polypeptide is that which is substantially free of contaminating materials from the material from which it was obtained, e.g. cellular materials, such as but not limited to cell debris, cell wall materials, membranes, organelles, the bulk of the nucleic acids, carbohydrates, proteins, and/or lipids present in cells. Thus, a polypeptide that is isolated includes preparations of a polypeptide having less than about 30%, 20%, 10%, 5%, 2%, or 1% (by dry weight) of cellular materials and/or contaminating materials. As used herein, the terms “purified” and “isolated” when used in the context of a polypeptide that is chemically synthesized refers to a polypeptide which is substantially free of chemical precursors or other chemicals which are involved in the syntheses of the polypeptide.

Polypeptides may be isolated and purified in accordance with conventional methods of recombinant synthesis or cell free protein synthesis. Methods which are well known to those skilled in the art can be used to construct expression vectors containing coding sequences and appropriate transcriptional/translational control signals. These methods include, for example, in vitro recombinant DNA techniques, synthetic techniques and in vivo recombination/genetic recombination. Alternatively, RNA capable of encoding the polypeptides of interest may be chemically synthesized. One of skill in the art can readily utilize well-known codon usage tables and synthetic methods to provide a suitable coding sequence for any of the polypeptides of the invention.

A “patient” for the purposes of the present invention includes both humans and other animals, particularly mammals, including pet and laboratory animals, e.g. mice, rats, rabbits, etc. Thus the methods are applicable to both human therapy and veterinary applications. In one embodiment the patient is a mammal, preferably a primate. In other embodiments the patient is human.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having cancer. Subjects may be human, but also include other mammals, particularly those mammals useful as laboratory models for human disease, e.g. mouse, rat, etc.

“In combination with”, “combination therapy” and “combination products” refer, in certain embodiments, to the concurrent administration to a patient of the agents described herein. When administered in combination, each component can be administered at the same time or sequentially in any order at different points in time. Thus, each component can be administered separately but sufficiently closely in time so as to provide the desired therapeutic effect.

“Concomitant administration” of active agents in the methods of the invention means administration with the reagents at such time that the agents will have a therapeutic effect at the same time. Such concomitant administration may involve concurrent (i.e. at the same time), prior, or subsequent administration of the agents. A person of ordinary skill in the art would have no difficulty determining the appropriate timing, sequence and dosages of administration for particular drugs and compositions of the present invention.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use.

A “therapeutically effective amount” means the amount that, when administered to a subject for treating a disease, is sufficient to effect treatment for that disease.

Production of Nanoparticle

Suitably to form the nanoparticle, a polymer may be dissolved into an organic phase (oil) that is emulsified with a surfactant or stabilizer (water). Hydrophobic drugs may be added directly to the oil phase, whereas hydrophilic drugs (water) may be first emulsified with the polymer solution prior to formation of particles. High intensity sonication can be used to facilitate the formation of small polymer droplets. The resulting emulsion is added to a larger aqueous phase and stirred for several hours, which allows the solvent to evaporate. Hardened nanoparticles can be collected and washed by centrifugation.

The concentration of polymer will usually be at least about 0.01 mg/ml, more usually at least about 0.1 mg/ml, at least about 1 mg/ml, and not more than about 100 mg/ml, usually not more than about 50 mg/ml. The ratio of active agents to polymer as a weight percent will vary, from around about 1:1000; 1:500; 1:100, 1:50; 1:10; 1:5, and the like.

Nanoparticles may have a controlled size, as appropriate for optimization of delivery of the biologically active agents. Usually the particle will have a diameter from about 50 nm, from about 100 nm, up to about 250 nm, up to about 500 nm, up to about 1 μm, up to about 2.5 μm, up to about 5 μm, and not more than about 10 μm in diameter. In some embodiments the nanoparticle size is from about 100 nm to about 500 nm in diameter, for example from about 100 nm to about 300 nm, from about 300 nm to about 2 μm, and the like. The nanoparticle optionally has a defined size range, which may be substantially homogeneous, where the variability may not be more than 100%, 50%, or 10% of the diameter. The sizes of particles can be altering by varying parameters, including the payload, the hydrophobicity of polymer, the type of organic solvent, organic solvent volume, solvent to polymer ratio, emulsifier, sonication intensity, centrifugation speed and time, and the like.

The nanoparticle may comprise a coating of any biologically compatible polymer. Biodegradable polymers useful as a coating may include hydroxyaliphatic carboxylic acids, either homo- or copolymers, such as poly(lactic acid), poly(glycolic acid), Poly(dl-lactide/glycolide, poly(ethylene glycol); polysaccharides, e.g. lectins, glycosaminoglycans, e.g. chitosan; celluloses, acrylate polymers, and the like. The coating may influence the rate of degradation of the nanoparticle after administration, targeting of the nanoparticle of the nanoparticle and the like.

Suitably, the nanoparticle may include approximately 4 mg of encapsulated ovalbumin protein.

Methods of Use

For the treatment of cancer, the nanoparticles can be administered as a single agent; Administration can be repeated as required, e.g. dosing 1, 2, 3, 4, 5, 6, 7, 8, 9, 10, 11, 12, etc. times, where the interval between doses can be, for example, 1 day, two days, 3 days, 4 days, 5 days, 6 days, 1 week, 2 weeks, 1 month, etc., and ranges therein, until such time as the desired effect is obtained. The initial dose(s) can be a priming dose, e.g. a lower dose than the therapeutic dose, or can be a higher dose than the therapeutic dose.

As used herein “cancer” includes any form of cancer, including but not limited to solid tumour cancers (e.g., lung, prostate, breast, bladder, colon, ovarian, pancreas, kidney, liver, glioblastoma, medulloblastoma, leiomyosarcoma, head & neck squamous cell carcinomas, melanomas, neuroendocrine; etc.) and liquid cancers (e.g., hematological cancers); carcinomas; soft tissue tumours; sarcomas; teratomas; melanomas; leukemias; lymphomas; and brain cancers, including minimal residual disease, and including both primary and metastatic tumours.

Histological types of carcinomas include adenocarcinoma, squamous cell carcinoma, adenosquamous carcinoma, large cell carcinoma, small cell carcinoma, and anaplastic carcinoma. Carcinomas may be found in skin, lungs, pancreas, mouth, throat, esophagus, stomach, colon, breast, prostate, bladder, kidney, anus, ovary, brain and liver, etc. Examples of carcinomas include, but are not limited to: adenocarcinoma (cancer that begins in glandular (secretory) cells), e.g., cancers of the breast, pancreas, lung, prostate, and colon can be adenocarcinomas; adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like.

### EXAMPLES

Ovalbumin (OVA) has been used in many scientific projects to demonstrate that a vaccine vehicle (or its adjuvant) can produce a specific immune response in a vaccine recipient. Due to its popularity as a scientific model, processing of OVA by immune and epithelial cells in C57/BL6 mice has been extensively characterised. Around ⅓ of all proteins present in a cell are degraded and presented on the cell surface bound to Major Histocompatability Complex (MHC) proteins (in humans referred to as HLA or Human Leukocyte antigens). The MHC proteins are separated into two classes, known as class I and class II. After processing, fragments of OVA will bind to either MHC class I or MHC class II. The class to which the OVA binds determines the immunological response. For example, presentation of the OVA on MHC class I (amino acid sequence of the OVA peptide fragment that does this is SIINFEKL) will be recognised by a subset of T-cells known as CD8+ T-cells. These cytotoxic CD8+ T cells are of significant interest as they can be trained to specifically recognise tumour cells displaying a particular antigen. Whilst the application has been demonstrated using OVA, it will be understood that this is a model for other immunogenic peptides.

Dendritic cells are professional antigen presenting cells which, once activated by antigen internalisation, migrate to lymph nodes to present foreign antigens to T-cells. Any CD8+ T-cells that recognise the antigen (for example OVA) will proliferate within and migrate from the lymph nodes and disseminate around the body. Any cells that are displaying the SIINFEKL antigen on MHC class I will then be killed by circulating CD8+ cytotoxic T-cells.

The nanoparticles of the present invention, protect the antigen from degradation before it reaches the target tumour and dendritic cells. The antigen (in the model system SIINFEKL) must be selected and provided such that dendritic cells present the antigen in the context of MHC I such that CD8+ cytotoxic cells are provided. Further the tumour epithelial cells must display the desired antigen (SIINFEKL) in the context of MHC I so they are targeted by cytotoxic T-cells.

There are significant differences between how dendritic cells (DCs) and epithelial cells process antigens; however, the inventors have realised that antigen delivery via a nanoparticle system allows delivery of antigens from an exogenous source (i.e. the peptide was not originally produced in the cell) and cross presentation can occur. Whilst in dendritic cells cross presentation is common, it does not occur commonly in epithelial cells.

The following in vitro work was undertaken as proof of principle for the present invention.

Dendritic Cells (JAWSII)

SIINFEKL and ovalbumin encapsulated nanoparticles were formed and an assessment of antigen presentation (Flow cytometry) was undertaken together with cell surface confocal microscopy analysis to confirm surface presentation of MHC:SIINFEKL

Co incubation of OT1 CD8+ T cells with pulsed dendritic cells was undertaken to demonstrate biological activity of the antigen.

It is considered that in vitro work demonstrates MHC presentation of the non-tumour protein payload, causing production of antigen specific T-cell populations, IFN-γ ELISA shows activity.

There will be regression of tumour with regular injection intervals (This would be direct injection at first followed by IV injection).

### Example 1

### Materials and Methods for Particle Formulation

A model peptide SIINFEKL was used in this example. SIINFEKL peptide (SEQ ID NO: 1) (ANASPEC, Fremont, USA) was resuspended in PBS to a final stock concentration of 1 mM and stored at 4° C.

Nanoparticle Formulation

20 mg of Resomer® RG 502H, Poly(D,L-lactide-co-glycolide) (PLGA) acid terminated (lactide:glycolide 50:50; Mw 7,000-17000) polymer (Sigma, UK) was dissolved in 1 mL of ice cold dichloromethane (DCM). Following this, 200 μL of 1 mM SIINFEKL (ANASPEC, Fremont, USA) was added directly to the DCM.

Using a 25 gauge needle, the DCM/Polymer/SI IN FEKL mix was injected directly into mL of 2.5% (w/v) poly vinyl alcohol (Sigma UK) in 50 mM 2-(N-morpholino)ethanesulfonic acid (MES) hydrate buffer (pH 5) ((MES Buffer: 9.5 g—MES hydrate, 1 L of Distilled water, adjusted to pH 5 and autoclaved). 2.5% (w/v) PVA in 50 mM MES hydrate buffer: 12.5 g PVA, 500 mL MES, (Heated to 60° C. and stirred for 6 hours until PVA had dissolved before adjustment of pH to 5 and autoclaving)) under constant stirring at 400 RPM. The emulsion formation was induced by sonication (Model 120 Sonic Dismembrator, Fisher Scientific, UK) for 90 seconds on ice (amplitude of 50%) and left stirring at 400 RPM overnight to allow the solvent to evaporate. The following day, three sequential wash steps were performed. Particles were spun at 16,000 g at 4° C. for 20 minutes in each step. After each step, the nanoparticle pellet was suspended in PBS and sonicated (amplitude of 30%) to ensure the pellet was fully re-suspended. After the final wash step, the particles were resuspended in PBS at a final concentration of 5 mg/mL ready for use.

### Example 2

### Presentation of SIINFEKL Peptide is Dependent on Particle Uptake

MC38 cells were seeded at 5×10⁴ in 6 well plates and left overnight to adhere in 2 mL of the appropriate growth medium. Following this, the cells were treated with 250 μg/mL of either acid terminated poly(lactic-co-glycolic acid) (PLGA) nanoparticles (NPs) or PLGA NPs encapsulating the SIINFEKL peptide (for cells treated at 4 degrees C., cells were maintained at 4° C. for 45 minutes prior to treatment). Following treatment, the cells were washed 3 times with sterile PBS (4° C. treatment group were washed on ice) and the cells incubated for a following 6 hours at 37° C. The surface expression of SIINFEKL was determined by flow cytometry.

### Example 3

MC38 cells were seeded at 5×10⁴ in 6 well plates and left overnight to adhere in 2 mL of the appropriate growth medium. Following this, the cells were treated with 250 μg/mL of either acid terminated poly(lactic-co-glycolic acid) (PLGA) nanoparticles (NPs) or PLGA NPs encapsulating the Ovalbumin protein. Free, pre-processed SIINFEKL was used as a positive control. Cells were incubated for 24 hours at 37° C. Following this, the cells were collected and incubated with a PE-conjugated antibody specific for MHC-I bound to the SIINFEKL peptide (Biolegend, USA). Next, presentation of the SIINFEKL peptide was assessed by flow cytometry.

### Example 4

### Confocal Microscopy of SIINFEKL Presented on MHC-I on the Cell Surface

To undertake confocal microscopy of SIINFEKL presented on MHC-I on the cell surface, MC38 cells can be seeded at 5×10⁴ in a 6 well plate and left overnight. The following day they can be treated with 500 μg of either PLGA nanoparticles (NPs) or PLGA NPs encapsulating the Ovalbumin protein. After incubation for 24 hours, the cells can be fixed in 4% paraformaldehyde for 10 minutes. Following 3 washes with PBS, the cells can be permeabilised with 0.2% PBS Tween for 10 minutes and washed a further 3 times. To prevent non-specific antibody binding, the cells can be incubated with a solution of 1% BSA in 0.1% PBS tween for 30 minutes. MHC-SIINFEKL specific antibody can then be added to the cells at a final concentration of 1:1000 and incubated for 1 hour at room temperature. After 3 washes with 0.1% PBS tween cells can be mounted onto DAPI containing ProLong™ Diamond Antifade Mountant (Thermo Fisher, UK) and images processed on a confocal microscope.

### Example 5

### SIINFEKL Biological Activity Assay

To undertake a SIINFEKL biological activity assay OT-I cells can be isolated from OT-1 mice using a spleen dissociation kit (Miltenyi, Germany), a single cell suspension can be formed according to the manufacturer's instructions and CD8⁺ cells enriched for using Dynabeads™ FlowComp™ Mouse CD8 Kit (Thermo Fisher, UK). Resulting CD8+ T-cells can be cultured and maintained. MC38 cells can then be seeded and treated. Following treatment, OT-1 CD8+ T-cells can be co-incubated with the MC38 cells for 24 hours. IFN γ secretion can be used to measure T-cell activity.

### Example 6

### HLA Immunoprecipitation (HLA-IP): Assessment of Haemagglutinin Cross Presentation in Human Epithelial Tumour Cells

To assess Haemagglutinin cross presentation in Human Epithelial Tumour cells, HCT116 and A549 cells can be seeded at 5×10⁶ in P140s and left overnight to adhere. The following day, PLGA NP or PLGA NP encapsulating recombinant haemagglutinin can be added to the cells and incubated at 37° C. for 24 hours. Following this, 75 μL of HLA/B/C conjugated Dynabeads® can be added to the sample and gently mixed to ensure total sample coverage. After incubation, the media can be aspirated and cells washed in PBS. The PBS can then removed and DISC IP buffer supplemented with protease inhibitors added directly onto the cells. Cells can be lysed for 30 minutes at 4° C. with gentle agitation. All procedures are conducted on ice or at 4° C. Cells can be scraped and collected in 2 mL tubes, and placed in a Dynamag™-2 magnetic rack. The flow through (i.e. anything not bound to the beads) can be transferred to a fresh 2 mL tube and kept on ice. The bound beads are then washed 5 times in 750 μL HLA elution buffer and after the final wash centrifuged and HLA elution buffer is removed. 100 μL of 2× loading buffer can be added and subsequently heated at 95° C. for 5 minutes to prepare the sample for mass spectroscopy.

### Example 7

### Assessment of HLA-Class I Epitopes Presented on Human Epithelial Tumour Cells

To reduce contamination of the sample with various cellular proteins, the HLA-IP sample can be resolved by running the samples on a 6% SDS-PAGE. The gel can be sliced at between 0 and 10 Kda to ensure all MHC I and II epitopes are isolated (between 8 and 14 amino acids respectively). Following this, proteins are reduced (10 mM DTT) at 56° C. for 1 hour and alkylated (55 mM iodacetamide) at 25° C. for 30 minutes followed by alternate washing with water and H₂O/50% acetonitrile (ACN). After trypsin digestion. Peptides can be extracted using 50% ACN/0.1% TFA followed by 100% CAN. These peptides can then be analysed using Liquid chromatography-mass spectrometry (LC/MS).

### Example 8

### Preparation of Lysosome Disrupting PLGA NPs

PLGA nanoparticles were prepared as previously described. Several lysosomal inhibitors/disruptors were encapsulated or coated into/onto the PLGA NP through addition of the drug to the organic phase of the composition. The lysosomal disruption agents used included (but was not limited to)—E64, Chloroquine, Leu-Leu-OME, Cathepsin inhibitors, Alum, Omeprozole, Mefloquine, Tafenoquine. Hydrophobic drugs were encapsulated as previously described, however, for hydrophilic drugs a water-in-oil-in-water (W/O/W) was used. The drug was dissolved in a water based solvent (such as PBS) and injected into 1 mL of ice cold dichloromethane. This was followed by a 30 second sonication (30% amplitude). This was then injected into the aqueous phase as previously described and sonicated to complete particle formulation.

Cells could also be pre-treated with drug and then the nanoparticles added.

### Example 9

### Lysosome Disruption Assay

Cells were seeded at 5×10⁴ in a 6 well plate and left overnight. The cells were then treated with PLGA-Lysosome disrupting agent formulations for 24 hours. Following this, the media was removed and the cells washed with PBS and detached from the wells using 1 mL of trypsin. The cells were collected in a falcon tube and centrifuged at 2400 RPM for 5 minutes. The supernatant was discarded and the pellet resuspended in PBS containing 1 μg/mL of acridine orange. After a 15 minute incubation at room temperature, staining intensity was measured by flow cytometry. Successful particles were then tested.

Flow cytometry was carried out on a BD Accuri™ C6 Plus Flow cytometer (BD Biosciences, San Diego, Calif., USA) and analysis was completed on BD Csampler™ Plus software (version 1.0.23.1).

DynaBeads® Antibody conjugation: HLA A/B/C specific antibody (Biolegend, UK) was conjugated to Dynabeads® (Thermo Fisher, UK) according to manufacturer's instructions. Due to the requirement for Mass spectroscopy, PBS was used in substitute for the SB buffer in the kit provided.

MC38 and A549 cells were cultured in DMEM (Sigma, UK) supplemented with 10% FCS (Thermo Fisher, UK) and 1% Pen/Strep (Thermo Fisher, UK). HCT116 cells were cultured in McCoys 5A (modified) medium (Thermo Fisher, Paisley, UK) supplemented with 10% FCS and 1% sodium pyruvate.

Although the invention has been particularly shown and described with reference to particular examples, it will be understood by those skilled in the art that various changes in the form and details may be made therein without departing from the scope of the present invention.

1. A composition for the delivery of a non-tumour associated target antigen to a cell, wherein the composition comprises.
a polymeric nanoparticle
a non-tumour associated target antigen payload
wherein the polymeric nanoparticle encapsulates the payload to provide the payload to a cancer cell such that the payload is substantially masked from antibody opsonisation and complement activation.

2. The composition of claim 1 wherein the polymeric nanoparticle comprises at least one polymer selected from the group consisting of
lactic acid polymers (PLA)
glycolic acid polymers (PLG); and
poly (lactic-co-glycolic) acid (PLGA).

3. The composition of claim 1 wherein the non-tumour associated payload is selected from a non-viral synthetic peptide.

4. The composition of claim 1 wherein the non-tumour associated payload is provided in combination with a lysosome perturbation agent, optionally wherein the lysosome perturbation agent is selected from at least one of Legumain or Cathepsin inhibitors, Alum, Omeprozole, Mefloquine, Tafenoquine, and Leu-Leu-OMe.

5. The composition of claim 1 wherein the non-tumour associated payload is selected from a viral protein, optionally selected from;
(a) Influenza virus coat protein derived from a serotypes including: A B and C but excluding D,
(b) coranvirus spike protein epitope, optionally QYIKWPWYI
(c) a haemagglutinin subtype or neuraminidase subtypes and/or
(d) a coat protein of the herpesviridae family, optionally Epstein Barr Virus proteins comprising constituents of the gHgLgp42 complex and LMP2a.

6. The composition of claim 1 provided in combination with at least one immune checkpoint inhibitor targeting the PD-L1/PD-1 and CTLA4 axis and/or to prevent inhibition of cancer specific CD8+ T-cells, optionally
a CTLA4 inhibitor, optionally selected from
Ipilimumab (Yervoy)
Tremelimumab, or
an anti PD-1 inhibitor, optionally selected from
Nivolumab (Opdivo)
Pembrolizumab (Keytruda)
Spartalizumab
Cemiplimab, or
an anti-PD-Ll inhibitor, optionally selected from
Atezolizumab
Avelumab
Durvalumab or
5-FU and Oxaliplatin (FOLFOX) with or without co-administration of Leucovorin, or other folinic acid derivatives or analogues,
5-FU and Irinotecan (FOLFIRI) with or without co-administration of Leucovorin, or other folinic acid derivatives or analogues,
5-FU with or without co-administration of Leucovorin, or other folinic acid derivatives or analogues, or
a combination of Oxaliplatin and Capecitabine (Xelox).

7. A method of treating cancer comprising the step of administering a therapeutically effective dose of the composition according to claim 1 to a subject in need thereof.

8. A method of treating cancer as claimed in claim 7 wherein the cancer causes a tumour and has an inflammatory phenotype.

9. A method of treating cancer as claimed in claim 7 wherein the cancer is melanoma; microsatellite stable (MSS) colorectal cancer CIMP Hi and Lo and CMS1 classified tumours; lung cancer, pancreatic cancer; renal cancer; or any other solid tumour cancer.

10. A method of treating cancer as claimed in claim 9 wherein the cancer is lung cancer selected from Squamous, Non-squamous, Non-Small Cell Lung Cancer and Small Cell lung cancer.

11. A method of treating cancer as claimed in claim 10 wherein the cancer is melanoma and the treatment is by intra-tumour administration.

12. A composition for use in the treatment of cancer, wherein the composition comprises
a polymeric nanoparticle
a non-tumour associated target antigen payload
wherein the polymeric nanoparticle encapsulates the payload to provide the payload to a cancer cell such that the payload is substantially masked from antibody opsonisation and complement activation.

13. The composition for use in the treatment of cancer of claim 12 wherein the polymeric nanoparticle comprises a polymer selected from the group consisting of
lactic acid polymers (PLA)
glycolic acid polymers (PLG); and
poly (lactic-co-glycolic) acid (PLGA).

14. The composition for use in the treatment of cancer of claim 12 wherein the non-tumour associated payload is selected from a non-viral synthetic peptide.

15. The composition for use in the treatment of cancer of claim 12 wherein the non-tumour associated payload is selected from a viral protein, optionally selected from;
(a) Influenza virus coat protein derived from a serotypes including: A B and C but excluding D,
(b) a haemagglutinin subtype or neuraminidase subtypes and/or
(c) a coat protein of the herpesviridae family, optionally Epstein Barr Virus proteins
comprising constituents of the gHgLgp42 complex and LMP2a.

16. The composition for use in the treatment of cancer of claim 12 provided in combination with at least one immune checkpoint inhibitor targeting the PD-L1/PD-1 and CTLA4 axis and or to prevent inhibition of cancer specific CD8+ T-cells, optionally
a CTLA4 inhibitor, optionally selected from
Ipilimumab (Yervoy)
Tremelimumab, or
an anti PD-1 inhibitor, optionally selected from
Nivolumab (Opdivo)
Pembrolizumab (Keytruda)
Spartalizumab
Cemiplimab,
an anti-PD-L1 inhibitor, optionally selected from
Atezolizumab
Avelumab
Durvalumab or
5-FU and Oxaliplatin (FOLFOX) with or without co-administration of Leucovorin, or other folinic acid derivatives or analogues,
5-FU and Irinotecan (FOLFIRI) with or without co-administration of Leucovorin, or other folinic acid derivatives or analogues,
5-FU with or without co-administration of Leucovorin, or other folinic acid derivatives or analogues, or
a combination of Oxaliplatin and Capecitabine (Xelox).
